(19) United States
(12) Patent Application Publication
(10) Pub. No.: US 2022/0378310 A1
(43) Pub. Date: Dec. 1, 2022

(54) DETECTING HEART RATES USING EYE-TRACKING CAMERAS

(71) Applicant: Microsoft Technology Licensing, LLC, Redmond, WA (US)

(72) Inventors: Jonathan Eric FOSTER, Kirkland, WA (US); Daniel Jonathan MCDUFF, Cambridge, MA (US)

(21) Appl. No.: 17/332,056

(22) Filed: May 27, 2021

(51) Int. Cl. A61B 5/024; G06F 3/01; G02B 27/01

(52) U.S. Cl. CPC A61B 5/02438; G06F 3/013; A61B 5/02416; G02B 27/0172; G02B 2027/0138; G02B 2027/014

(57) ABSTRACT

A head-mounted device includes one or more eye-tracking cameras and one or more computer-readable hardware storage devices having stored thereon computer-executable instructions, including a machine-learned artificial intelligence (AI) model. The head-mounted device is configured to cause the one or more eye-tracking cameras to take a series of images of one or more areas of skin around one or more eyes of a wearer, and use the machine-learned AI model to analyze the series of images to extract a photoplethysmography waveform. A heart rate is then detected based on the photoplethysmography waveform.

### BACKGROUND

A heart rate (HR) monitor is a personal monitoring device that allows one to measure and/or display heart rate in real time or record the heart rate for later study. It is sometimes used to gather heart rate data while performing various types of physical exercise. Medical heart rate monitoring devices used in hospitals usually include wired multiple sensors. One type of commonly used HR monitor that uses electrical sensors to measure heart rate is referred to as electrocardiography (also referred to as ECG or EKG). The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter.

The embodiments described herein are related to detecting heart rates using eye-tracking cameras of head-mounted devices. A head-mounted device includes one or more eye-tracking cameras, one or more processors, and one or more computer-readable hardware storage devices. The computer-readable hardware storage devices store computer-executable instructions, including a machine-learned AI model. The computer-executable instructions are structured such that, when executed by the one or more processors, the head-mounted device is configured to cause the one or more eye-tracking cameras to take a series of images of an area of skin around one or more eyes of a wearer. The head-mounted device is further configured to use the machine-learned AI model to analyze the series of images to extract a photoplethysmography (PPG) waveform and detect a heart rate based on the PPG waveform.

The embodiments described herein are also related to training a machine-learned AI model for detecting heart rates based on a series of images taken by one or more eye-tracking cameras of a head-mounted device. Training the machine-learned AI model includes providing a machine learning network configured to train an AI model based on images taken by eye-tracking cameras of head-mounted devices. A plurality of series of images of one or more areas of skin around one or more eyes of the wearer is taken by the one or more eye-tracking cameras of the head-mounted device as training data. The plurality of series of images is then sent to a machine learning network to train a machine-learned AI model in a particular manner, such that the machine-learned AI model is trained to extract a PPG waveform and detect a heart rate based on the PPG waveform.

Additional features and advantages will be set forth in the description which follows, and in part will be obvious from the description or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not, therefore, to be considered to be limiting in scope, embodiments will be described and explained with additional specificity and details through the use of the accompanying drawings in which:

FIG. 1 illustrates an example of an architecture of a head-mounted device, in which principles described herein are implemented;

FIGS. 2A and 2B illustrate an example of a simplified structure of a head-mounted device including one or more eye-tracking cameras;

FIG. 3A illustrates an example of an embodiment of training an artificial intelligence (AI) model configured to generate photoplethysmography (PPG) waveform based on images taken by one or more eye-tracking cameras;

FIG. 3B illustrates another example of an embodiment of training an artificial intelligence (AI) model configured to generate photoplethysmography (PPG) waveform based on images taken by one or more eye-tracking cameras;

FIG. 4A illustrates an example of an embodiment of using a machine-trained AI model shown in FIG. 3A to detect a heart rate based on images generated by one or more eye-tracking cameras of a head-mounted device;

FIG. 4B illustrates an example of an embodiment of using a machine-trained AI model shown in FIG. 3B to detect a heart rate based on images generated by one or more eye-tracking cameras of a head-mounted device;

FIG. 5 illustrates a flowchart of an example of a method for training a machine-learned AI model configured to detect heart rates based on images taken by one or more eye-tracking cameras;

FIG. 6 illustrates a flowchart of an example of a method for using a machine-learned AI model to detect heart rates based on images taken by one or more eye-tracking cameras;

FIG. 7 illustrates a flowchart of an example of a method for segmenting out data generated during noisy time windows; and

FIG. 8 illustrates an example computing system in which the principles described herein may be employed.

### DETAILED DESCRIPTION

The principles described herein are related to detecting heart rates using eye-tracking cameras of head-mounted devices. A head-mounted device includes one or more eye-tracking cameras, one or more processors, and one or more computer-readable hardware storage devices. The computer-readable hardware storage devices store computer-executable instructions, including a machine-learned AI model. The computer-executable instructions are structured such that, when executed by the one or more processors, the head-mounted device is configured to cause the one or more eye-tracking cameras to take a series of images of an area of skin around one or more eyes of a wearer. The head-mounted device is further configured to use the machine-learned AI model to analyze the series of images to extract a photoplethysmography (PPG) waveform and detect a heart rate based on the PPG waveform.

In some embodiments, at least one of the one or more eye-tracking cameras is an infrared camera. In some embodiments, the head-mounted device further includes one or more infrared light sources configured to emit infrared light at the one or more areas of skin around the one or more eyes of the wearer.

In some embodiments, the head-mounted device further includes an inertial measurement unit configured to detect head motion of the wearer, and the head-mounted device is further configured to remove at least a portion of noise artifacts generated by the head motion from the PPG waveform. In some embodiments, the inertial measurement unit includes at least one of (1) an accelerometer, (2) a gyroscope, and/or (3) a magnetometer.

In some embodiments, data related to the head motion of the wearer is also input into the machine-learned AI model, causing the machine-learned AI model to cancel out at least a portion of noise artifacts generated by the head motion from the PPG waveform. In some embodiments, the head-mounted device is further configured to process data generated by the inertial measurement unit to identify one or more frequency bands of the noise artifacts generated by the head motion and filter the one or more frequency bands out of the PPG waveform.

In some embodiments, the head-mounted device is further configured to determine whether a period of time is too noisy based on data generated by the inertial measurement unit during the period. In response to determining that the period is too noisy, data generated during the period is segmented out. Such data includes one or more images among the series of images taken during the period. In some embodiments, determining whether the period of time is too noisy includes determining a standard deviation of values obtained from the inertial measurement unit during the period of time. When the standard deviation is greater than a predetermined threshold, it is determined that the period of time is too noisy. When the standard deviation is no greater than the predetermined threshold, it is determined that the period of time is not too noisy.

In some embodiments, the head-mounted device is further configured to allow a wearer to perform a calibration operation to improve the machine-learned AI model based on the individual wearer. The calibration includes (1) detecting a first heart rate dataset of the wearer based on a series of images taken by the one or more eye-tracking cameras and the machine-learned AI model, and (2) detecting a second heart rate dataset of the wearer via a heart rate monitor while the series of images are taken. The second heart rate dataset is then used as feedback to calibrate the machine-learned AI model.

In some embodiments, the head-mounted device further includes one or more displays configured to display one or more images in front of one or more eyes of the wearer. The head-mounted device is further configured to remove at least a portion of noise artifacts generated by the one or more displays from the PPG waveform. In some embodiments, data related to the one or more images displayed on the one or more displays is input into the machine-learned AI model, causing the machine-learned AI model to cancel out at least a portion of noise artifacts generated by the one or more displays.

In some embodiments, the head-mounted device is further configured to determine whether a period of time is too noisy based on data generated by the one or more displays during the period. In response to determining that the period is too noisy, data generated during the period is segmented out. Such data includes one or more images among the series of images taken during the period. In some embodiments, determining whether the period of time is too noisy includes determining a standard deviation of values obtained from the display unit during the period of time. When the standard deviation is greater than a predetermined threshold, it is determined that a predetermined time window is too noisy. When the standard deviation is no greater than the predetermined threshold, it is determined that the predetermined time window is not too noisy.

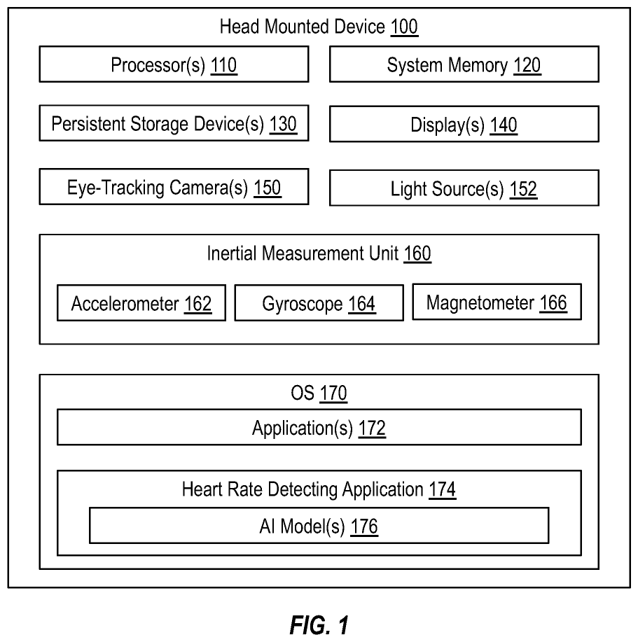

FIG. 1 illustrates an example of an architecture of a head-mounted device 100. The head-mounted device 100 includes one or more processors 110, one or more system memories 120, and one or more persistent storage devices 130. In some embodiments, the head-mounted device 100 also includes one or more displays 140 and/or one or more eye-tracking cameras 150. The one or more displays 140 are configured to display one or more images in front of the eyes of a wearer. The one or more eye-tracking cameras 150 are configured to track the eye movement of the wearer.

In some embodiments, the head-mounted device 100 also includes one or more light sources 152 configured to illuminate light onto one or more areas around the one or more eyes of the wearer to help the one or more eye-tracking cameras 150 to better tracking the eye movement of the wearer. In some embodiments, the eye-tracking camera(s) 150 are infrared (IR) cameras, and the light source(s) 152 are IR light source(s). In some embodiments, the head-mounted device 100 also includes an inertial measurement unit 160 configured to measure the wearer's head motion, such as (but not limited to) speed, acceleration, angular rate, and/or an orientation of the wearer's head. In some embodiments, the inertial measurement unit 160 includes at least one of an accelerometer 162, a gyroscope 164, and/or a magnetometer 166.

In some embodiments, an operating system (OS) 170 is stored in the one or more persistent storage devices 130 and loaded in the one or more system memories 120. In some embodiments, one or more applications 172 are also stored in the one or more persistent storage devices 130 and loaded in the one or more system memories 120. In the embodiments described herein, among the one or more applications, there is a heart rate detecting application 174 configured to detect a heart rate of a wearer. In particular, the heart rate detecting application 174 includes one or more machine-learned artificial intelligence (AI) models 176 configured to extract a PPG waveform based on the images taken by the eye-tracking cameras 150 and detect a heart rate based on the PPG waveform.

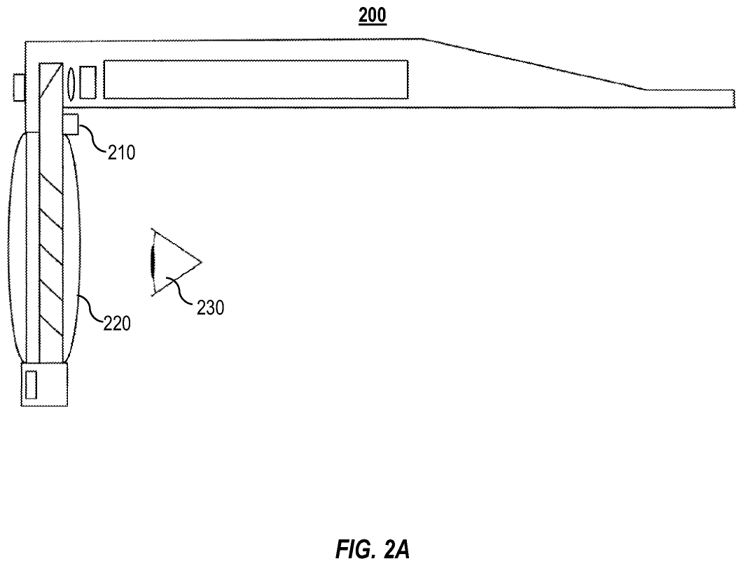

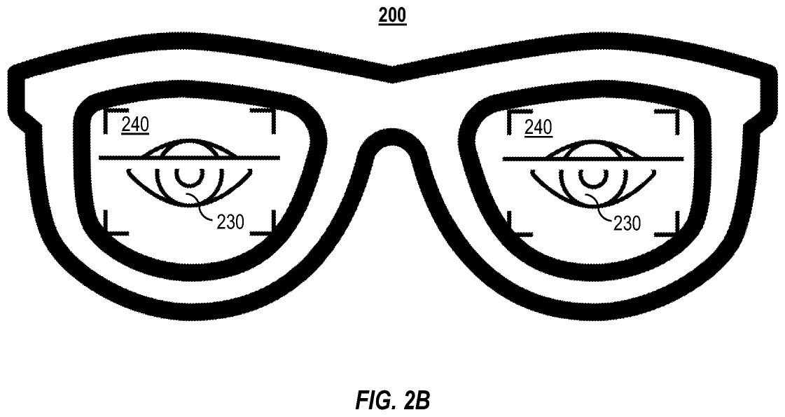

FIG. 2A illustrates a side view of a simplified example structure of a head-mounted device 200, which corresponds to the head-mounted device 100 of FIG. 1. As illustrated, the head-mounted device 200 includes one or more displays 220 and one or more eye-tracking cameras 210. The one or more displays 220 are configured to display images in front of one or more eyes 230 of a wearer, and the one or more eye-tracking cameras 210 are configured to track the movements of eye(s) 230 of the wearer. Notably, when the eye-tracking camera(s) 210 track movements of eye(s) 230 of the wearer, they are configured to capture images of area(s) of skin surrounding the eye(s) 230.

FIG. 2B illustrates a front view of the simplified example structure of the head-mounted device 200. As illustrated, the eyes 230 of the wearer and areas of skin 240 surrounding the eyes 230 are captured by the one or more eye-tracking cameras 210. The images taken by the eye-tracking cameras 210 can be used not only to track eye movements of the wearer but also to detect heart rates of the wearer.

The principles described herein are also related to training a machine-learned AI model for detecting heart rates based on a series of images taken by one or more eye-tracking cameras of a head-mounted device. Training the machine-learned AI model (also referred to as the AI model) includes providing a machine learning network configured to train an AI model based on images taken by eye-tracking cameras of head-mounted devices at a computing system. In some embodiments, the computing system is the head-mounted device 100, 200. In some embodiments, the computing system is a separate computing system that is different from the head-mounted device 100, 200.

During the training of the AI model, a plurality of series of images of one or more areas of skin around one or more eyes of the wearer is taken by the one or more eye-tracking cameras of the head-mounted device as training data. The plurality of series of images is then sent to a machine learning network to train a machine-learned AI model in a particular manner, such that the machine-learned AI model is trained to extract a PPG waveform from images taken by the eye-tracking cameras and detect a heart rate based on the PPG waveform.

In some embodiments, the machine learning network is an unsupervised network that trains the machine-learned AI model based on unlabeled image data. In some embodiments, the machine learning network is a supervised network that trains the AI model based on labeled image data. In some embodiments, the method further includes gathering a plurality of heart rate datasets via a heart rate monitor simultaneously when the plurality of series of images is gathered. The plurality of series of images is then labeled with the plurality of heart rate datasets. The plurality of series of images that are labeled with the plurality of heart rate datasets are then used as training data to train the machine-learned AI model.

In some embodiments, each image in each series of images includes a plurality of pixels. Each pixel corresponds to a color value. The method further includes, for each image in each series of images, computing an average value based on color values corresponding to a plurality of pixels in an image. The machine learning network is used to train the AI model configured to extract a PPG waveform based on average values of images in the plurality of series of images.

In some embodiments, the head-mounted device further includes an inertial measurement unit configured to detect the head motion of the wearer. A plurality of datasets associated with the head motion of the wearer is gathered by the inertial measurement unit. The plurality of datasets associated with the head motion of the wearer is also used as training data in training the AI model, such that the AI model is trained to cancel out at least a portion of noise artifacts generated by head motions.

In some embodiments, the head-mounted device further includes one or more displays configured to display images in front of one or more eyes of the wearer. A plurality of datasets associated with the images displayed by the one or more displays is also gathered. The plurality of datasets associated with the images displayed by the one or more displays is also used as training data in training the AI model, such that the AI model is trained to cancel out at least a portion of noise artifacts generated by the images displayed on the one or more displays.

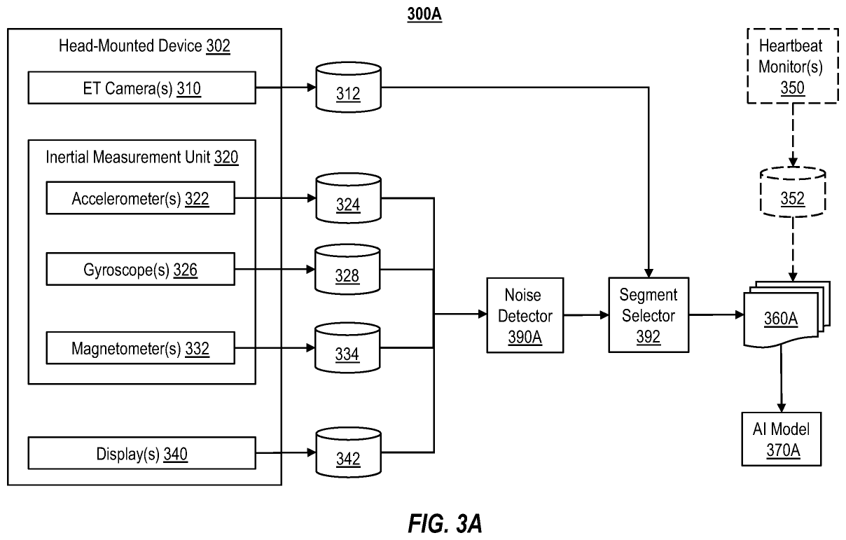

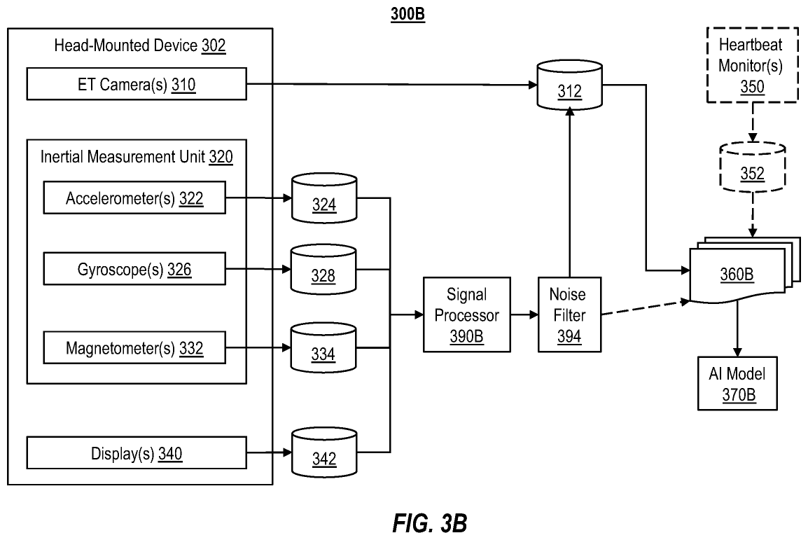

FIGS. 3A-3B illustrate examples of embodiments 300A and 300B for training an AI model 370A, 370B. Referring to FIG. 3A or 3B, data generated by one or more eye-tracking camera(s) 310 of a head-mounted device 302 is used as training data. The training data is sent to a machine learning network 360A, 360B to train an AI model 370A configured to detect heart rate based on images captured by the eye-tracking camera(s) 310. In some embodiments, the machine learning network 360A, 360B is a machine learning neural network. In some embodiments, the machine learning network 360A, 360B is a machine learning convolutional neural network. As illustrated, the head-mounted device 302 corresponds to the head-mounted device 100, 200 of FIGS. 1 and 2A-2B. The eye-tracking camera(s) 310 of the head-mounted device 302 are configured to capture a plurality of series of images 312. Each series of images is taken continuously in a time period, such as 30 seconds, 60 seconds, etc.

In some embodiments, the machine learning network 360A, 360B is an unsupervised training network, which uses unlabeled images 312 to generate an AI model 370A, 370B. In some embodiments, the machine learning network 360A, 360B is a supervised training network, which uses a plurality of heartbeat datasets 352 generated by one or more heartbeat monitor(s) 350 as feedback. In some embodiments, the training method is an unsupervised signal processing method. In some embodiments, the plurality of heartbeat datasets 352 are generated while the eye-tracking camera(s) 310 are taking the plurality of series of images 312, and the plurality of series of images 312 are labeled or paired with the plurality of heartbeat datasets 352. The labeled dataset pairs, including the plurality of series of images 312 and the plurality of heartbeat datasets 352, are used as training data to train the AI model 370A, 370B.

Notably, each image taken by the eye-tracking camera(s) includes a plurality of pixels, and each of the plurality of pixels is represented by a value. In some embodiments, the values of the plurality of pixels in each image are averaged to generate a mean value of the image, and a series of images would result in a series of mean values. In some embodiments, the machine learning network 360A, 360B is configured to identify patterns based on a plurality of series of mean values corresponding to the plurality of series of images 312 to train an AI model with or without labeling the image data 312.

Such training methods and the trained model 370A, 370B might work sufficiently well when the wearer is sitting still and not moving. However, when the wearer is moving, such as playing a video game and/or watching a video, noise artifacts would be introduced due to the movement of the wearer and/or the display of the head-mounted device 302.

The principles described herein introduce several different solutions to solve the above-described problems. Referring to FIG. 3A, in some embodiments, data associated with head motion 324, 328, 334 and/or data associated with the display(s) 340 are also used as training data to train the AI model 370A. As illustrated, in some embodiments, data associated with head motion is obtained via an inertial measurement unit 320, which corresponds to the inertial measurement unit 160 of FIG. 1. The inertial measurement unit 320 includes at least one of one or more accelerometer(s) 322, one or more gyroscope(s) 326, one or more magnetometer(s) 332. As illustrated, the accelerometer(s) 322 are configured to generate a first plurality of motion datasets 324, the gyroscope(s) 326 are configured to generate a second plurality of motion datasets 328, and the magnetometer(s) 332 are configured to generate a third plurality of motion datasets 334. In some embodiments, the motion datasets 324, 328, 334 are used as training data (in addition to the images taken by the eye-tracking cameras 310) to train the AI model 370A, such that the AI model 370A is trained to cancel at least a portion of the noise artifacts generated by the head motion.

Additionally, when the wearer is watching a video or play a game, the images generated by the display 340 of the head-mounted device 302 may also create noise artifacts. In some embodiments, dataset 342 associated with display(s) 340 is also used as training data to train the AI model 370A, such that the AI model is also trained to cancel at least a portion of the noise artifacts generated by the display(s) 340.

In some cases, the noise artifacts generated by the head motion and/or display(s) 340 may be too much, such that the noise artifacts cause the images generated by the eye-tracking camera(s) 310 to be unusable for detecting heart rates. For example, when the wearer's head is moving rapidly, or the display(s) 340 is displaying fast-moving images, such rapid head movement and fast-moving images cause the images captured by the eye-tracking camera(s) 310 to be too noisy to extract PPG waveforms.

To address the above-described problems, in some embodiments, a noise detector 390A is further implemented to determine whether the noise artifacts generated by the head motion and/or display(s) 340 are too significant. In other words, whether the images captured by the eye-tracking camera(s) 310 during a period of time are too noisy, or whether the period of time is too noisy. Different methods may be implemented to determine whether a given period of time is too noisy.

In some embodiments, in a given period of time, the datasets 324, 328, 334, and/or 342 captured by the inertial measurement unit 320 and/or the display(s) 340 are computed to generate one or more standard deviations. When at least one of the standard deviations is greater than a predetermined threshold, the noise detector 390A determines that the period is too noisy. When the period is determined to be too noisy, the data generated during that period is segmented out. In some embodiments, a segment selector 392 is implemented to segment out the data generated during periods that are too noisy. Such data includes the images 312 taken by the eye-tracking camera(s) 310, datasets 324, 328, 334 generated by the inertial measurement unit 320, and/or dataset 342 generated by the display(s) 340. As such, only the data generated during periods that are not too noisy is used as training data to train the AI model 370A.

FIG. 3B illustrates another embodiment for removing noise artifacts generated by head motion and display(s) 340. As illustrated in FIG. 3B, a signal processor 390B is implemented to process the data associated with the inertial measurement unit 320 and the display(s) 340. In some embodiments, the signal processor 390B is configured to determine one or more frequency bands of the noise artifacts associated with the inertial measurement unit 320 and the display(s) 340. Based on the detected noise, a noise filter 394 is generated. The noise filter 394 is configured to filter out the one or more frequency bands of the noise artifacts. In some embodiments, the noise filter 394 is applied to the plurality of series of images 312 generated by the eye-tracking camera(s) 310. In some embodiments, the noise filter 394 is sent to the machine learning network 360B to filter data processed or partially processed by the machine learning network 360B. In some embodiments, a segment selector (not shown) is also implemented after the signal processor 390B to segment out the training datasets generated during particular periods that are too noisy.

Once the AI model 370A, 370B is sufficiently trained, the AI model 370A, 370B is provided to a head-mounted device 100, 200, such that the head-mounted device 100, 200 can detect heart rates of a wearer based on images captured by the eye-tracking cameras 150, 210. In some embodiments, the AI model 370A, 370B is deployed onto the head-mounted device 100, 200. In some embodiments, the AI model 370A, 370B is provided as a cloud service, and the head-mounted device 100, 200 sends the images captured by the eye-tracking camera 150 to the cloud service, which in turn performs computations using the AI model 370A, 370B to determine the heart rates of the wearer.

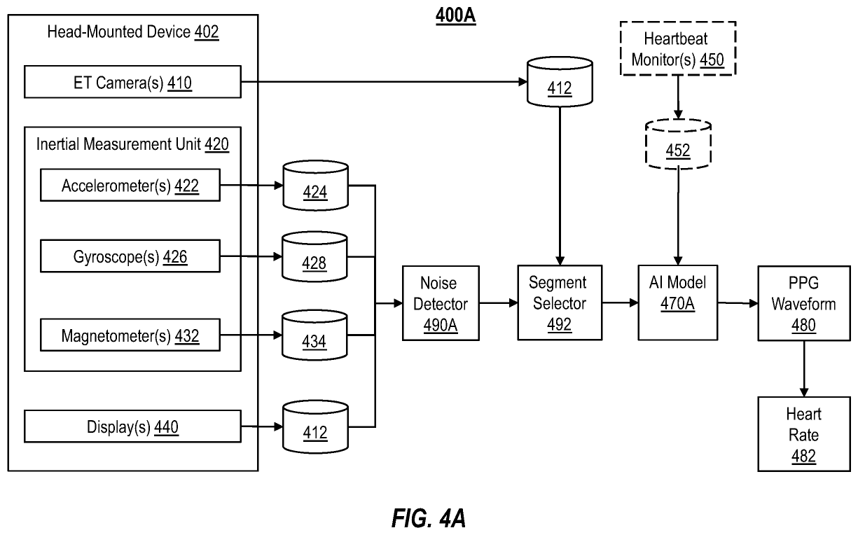

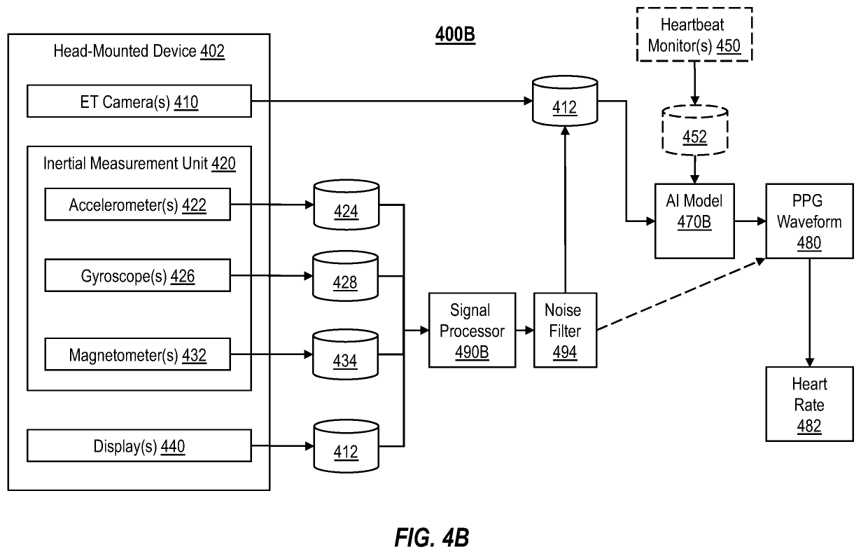

FIG. 4A illustrates an example of an embodiment 400A, in which a machine-trained AI model 470A (corresponding to the AI model 370A) is provided to a head-mounted device 402 (corresponding to the head-mounted device 100, 200, and/or 302). The head-mounted device 402 includes one or more eye-tracking cameras 410 configured to capture a series of images of one or more areas of skin around the one or more eyes of a wearer. In some embodiments, the machine-learned AI model 470A is configured to generate a PPG waveform 480 and detect a heart rate 482 of the wearer based on the PPG waveform.

In some embodiments, the head-mounted device 402 also includes an inertial measurement unit 420, which includes at least one of one or more accelerometer(s) 422, one or more gyroscope(s) 426, and/or one or more magnetometer(s) 432. In some embodiments, data generated by the inertial measurement unit 420 is also input into the machine-learned AI model 470A, and the machine-learned AI model 470A is configured to cancel out at least a portion of noise artifacts associated with the head motion of the wearer based on the data generated by the inertial measurement unit 420 (i.e., datasets 424, 428, 434).

In some embodiments, the head-mounted device 402 also includes one or more displays 440, which displays one or more images in front of one or more eyes of the wearer. In some embodiments, data associated with images 412 displayed on the display(s) 440 is also input into the machine-learned AI model 470A, and the machine-learned AI model 470A is further configured to cancel out at least a portion of noise artifacts associated with the images displayed on the display(s) 440.

In some embodiments, a noise detector 490A (which corresponds to the noise detector 390A) and a segment selector 492 (which corresponds to the segment selector 392) are also provided with the AI model 470A to the head-mounted device 402. The noise detector 490A is configured to determine whether the head motion and/or the display(s) 440 are creating too much noise during a given period. If the period is too noisy, the segment selector 492 is configured to segment out the data generated during the period, such as images taken by the eye-tracking camera(s) 410, data generated by the inertial measurement unit 420, and/or images displayed at the display(s) 440 during the period. As such, only data generated during the periods that are not too noisy is input to the AI model 470A to generate the PPG waveform 480, which is then used to detect a heart rate.

FIG. 4B illustrates an example of an embodiment 400B, in which a machine-trained AI model 470B (corresponding to the AI model 370B) is provided to a head-mounted device 402 (corresponding to the head-mounted device 100, 200, and/or 302). As illustrated in FIG. 4B, the data generated by the inertial measurement unit 420 and the display(s) 440 is sent to a signal processor 490B. In some embodiments, the signal processor 490B is configured to process the data generated by the inertial measurement unit 420 and the display(s) 440 to obtain one or more frequency bands of the noise artifacts. Based on the one or more frequency bands of the noise artifacts, a noise filter 494 is generated to filter out the one or more frequency bands of the noise artifacts from the images captured from the eye-tracking camera(s) 410 or from the PPG waveform 480 generated by the AI model 470B. The PPG waveform 480 is then processed to detect a heart rate 482.

In some embodiments, the machine-learned model 470A, 470B can further be calibrated based on individual wearers using a separate heartbeat monitor 450. For example, the heartbeat monitor 450 may be a fitness tracker, a watch, and/or a medical heartbeat monitor configured to track the heartbeats of a wearer. The dataset 452 generated by the heartbeat monitor 450 can be sent to the machine-learned AI model 470A, 470B to further calibrate and improve the machine-learned model 470A, 470B base on individual wearers of the head-mounted device 402.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

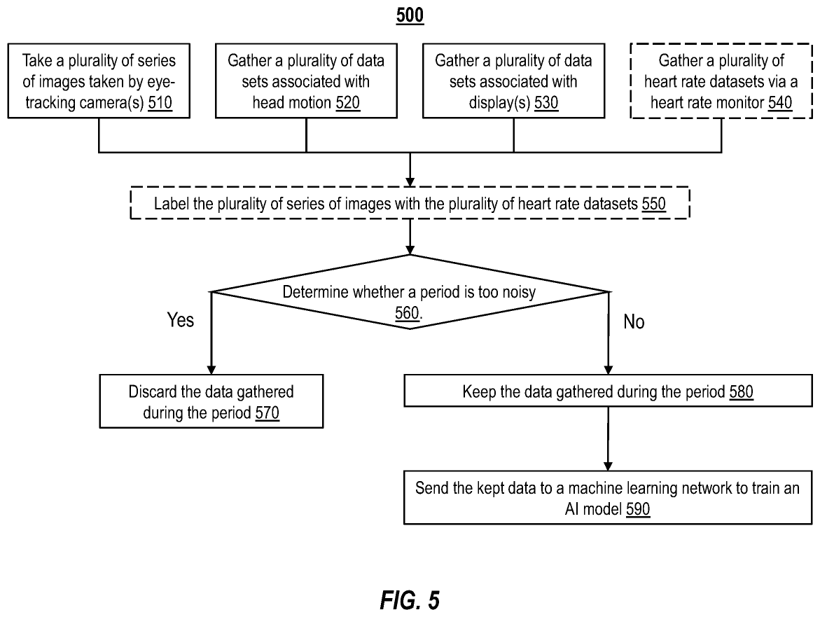

FIG. 5 illustrates a flowchart of an example method 500 for training an AI model for detecting heartbeat using a machine learning network. In some embodiments, the training of the AI model may be performed at a head-mounted device. Alternatively, the training of the AI model may be performed at a separate computing system or at a combination of the head-mounted device and the separate computing system. The method 500 includes taking a plurality of series of images of areas of skin around eye(s) of a wearer by one or more eye-tracking camera(s) of a head-mounted device (act 510). In some embodiments, the method 500 further includes gathering a plurality of data associated with the head motion of the wearer (act 520) and/or gathering a plurality of datasets associated with one or more display(s) (act 530).

In some embodiments, the machine learning network is an unsupervised learning network that uses unlabeled data as training data. Alternatively, the machine learning network is a supervised learning network that uses labeled data as training data. When a supervised learning network is used, the method 500 further includes gathering a plurality of heart rate datasets via a heart rate monitor (act 540) and labeling the plurality of series of images taken by the eye-tracking camera(s) with the heart rate datasets (act 550).

In some embodiments, method 500 further includes determining whether a given period is too noisy (act 560). If the period is too noisy, data gathered during the period is discarded (act 570). If the period is not too noisy, data gathered during the period is kept (act 580), and the kept data is sent to a machine learning network (which may be unsupervised or supervised) to train an AI model (act 590).

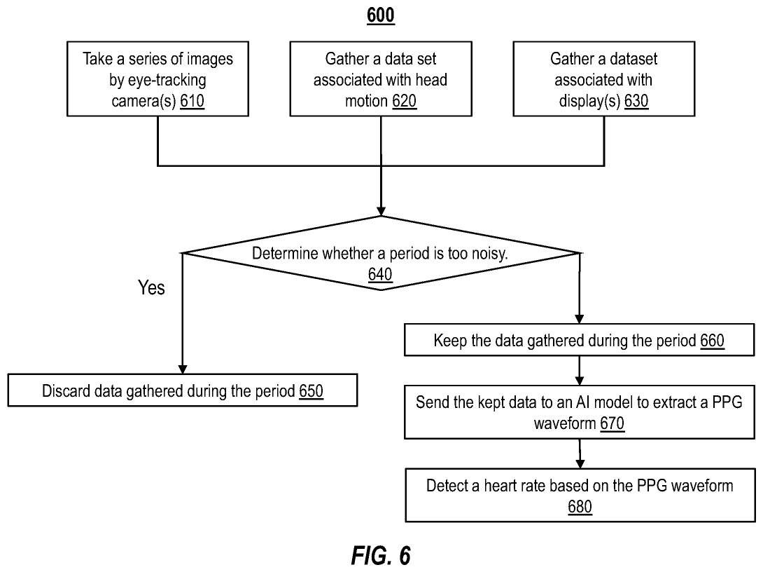

FIG. 6 illustrates a flowchart of an example of a method 600 for using a machine-learned AI model to detect a heart rate based on images taken by one or more eye-tracking camera(s) of a head-mounted device. The method 600 includes taking a series of images by one or more eye-tracking camera(s) (act 610). In some embodiments, the method 600 further includes gathering a dataset associated with head motion (act 620) and/or gathering a dataset associated with display(s) (act 630). In some embodiments, the method 600 further includes determining whether a given period is too noisy (act 640). If the period is determined to be too noisy, data gathered during the period is discarded (act 650); otherwise, data gathered during the period is kept (act 660). The kept data is then sent to a machine-trained AI model to extract a PPG waveform (act 670). Based on the PPG waveform, a heart rate is then detected (act 680).

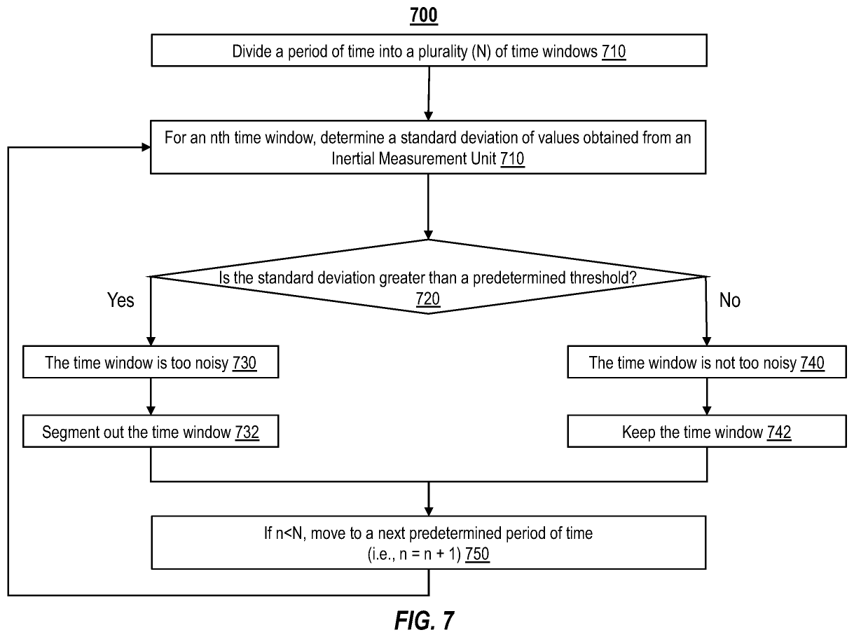

FIG. 7 illustrates a flowchart of an example method 700 for determining whether a given period is noisy, and segmenting data associated with noisy periods, which corresponds to acts 560, 570, 580 of FIG. 5 or acts 640, 650, 660 of FIG. 6. The method 700 includes dividing a period of time into a plurality of (N) time windows (act 710), where N is a natural number. The period of time may be any length that is sufficient to identify a heart rate of a wearer, such as 30 seconds, 1 minute, etc. Each time window has a predetermined size, such as 0.5 seconds, 1 second, 2 seconds, etc. The method 700 also includes, for an n^{th} time window, determining a standard deviation of values obtained from an inertial measurement unit during the n^{th} time window (act 710), where n is a natural number and n<=N. For example, in some embodiments, initially, n=1, and a standard deviation of values obtained from an inertial measurement unit is determined for the first time window among the plurality of N time windows.

The method 700 further includes determining whether the standard deviation is greater than a predetermined threshold (act 720). If the answer is yes, it is determined that the time window is noisy (act 730), and data generated during the time window is segmented out (act 732). On the other hand, if the answer is no, it is determined that the time window is not too noisy (act 740), and data generated during the time window is kept (act 742) as input of the machine learning network 360A, 360B or input of machine-learned AI model 470A, 470B. Next, if n<N, n=n+1, and the process repeats again based on data obtained during a next time window (act 750). For example, after it is determined whether data generated during the first time window is to be segmented out or kept, a second time window (i.e., n=2) is considered, and acts 710, 720, 730, 732, (or 740, 742), and 750 are repeated again based on n=2. In some embodiments, the acts 710, 720, 730, 732, (or 740, 742), and 750 repeat as many times as needed until each of the plurality of N time windows are analyzed.

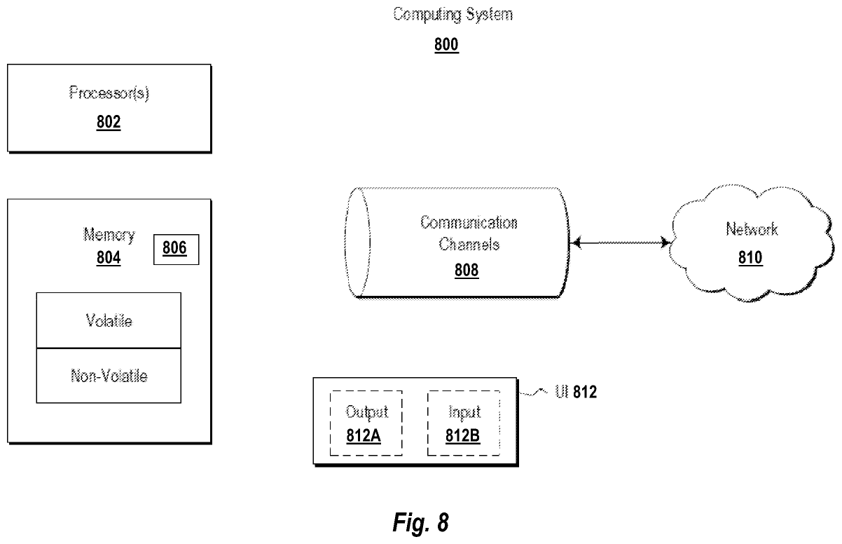

Finally, because the principles described herein may be performed in the context of a computing system (for example, each of the head-mounted device 100, machine learning network 360A, 360B may include or be implemented at one or more computing system) some introductory discussion of a computing system will be described with respect to FIG. 8.

Computing systems are now increasingly taking a wide variety of forms. Computing systems may, for example, be handheld devices, appliances, laptop computers, desktop computers, mainframes, distributed computing systems, data centers, or even devices that have not conventionally been considered a computing system, such as wearables (e.g., glasses). In this description and in the claims, the term “computing system” is defined broadly as including any device or system (or a combination thereof) that includes at least one physical and tangible processor, and a physical and tangible memory capable of having thereon computer-executable instructions that may be executed by a processor. The memory may take any form and may depend on the nature and form of the computing system. A computing system may be distributed over a network environment and may include multiple constituent computing systems.

As illustrated in FIG. 8, in its most basic configuration, a computing system 800 typically includes at least one processing unit 802 and memory 804. The at least one processing unit 802 may include a general-purpose processor and may also include a field-programmable gate array (FPGA), an application-specific integrated circuit (ASIC), or any other specialized circuit. The memory 804 may be physical system memory, which may be volatile, non-volatile, or some combination of the two. The term “memory” may also be used herein to refer to non-volatile mass storage such as physical storage media. If the computing system is distributed, the processing, memory and/or storage capability may be distributed as well.

The computing system 800 also has thereon multiple structures often referred to as an “executable component”. For instance, memory 804 of the computing system 800 is illustrated as including executable component 806. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such a structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component”.

The term “executable component” is also well understood by one of ordinary skill as including structures, such as hardcoded or hardwired logic gates, that are implemented exclusively or near-exclusively in hardware, such as within a field-programmable gate array (FPGA), an application-specific integrated circuit (ASIC), or any other specialized circuit. Accordingly, the term “executable component” is a term for a structure that is well understood by those of ordinary skill in the art of computing, whether implemented in software, hardware, or a combination. In this description, the terms “component”, “agent”, “manager”, “service”, “engine”, “module”, “virtual machine” or the like may also be used. As used in this description and in the case, these terms (whether expressed with or without a modifying clause) are also intended to be synonymous with the term “executable component”, and thus also have a structure that is well understood by those of ordinary skill in the art of computing.

In the description above, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied in one or more computer-readable media that form a computer program product. An example of such an operation involves the manipulation of data. If such acts are implemented exclusively or near-exclusively in hardware, such as within an FPGA or an ASIC, the computer-executable instructions may be hardcoded or hardwired logic gates. The computer-executable instructions (and the manipulated data) may be stored in the memory 804 of the computing system 800. Computing system 800 may also contain communication channels 808 that allow the computing system 800 to communicate with other computing systems over, for example, network 810.

While not all computing systems require a user interface, in some embodiments, the computing system 800 includes a user interface system 812 for use in interfacing with a user. The user interface system 812 may include output mechanisms 812A as well as input mechanisms 812B. The principles described herein are not limited to the output mechanisms 812A or input mechanisms 812B as such will depend on the nature of the device. However, output mechanisms 812A might include, for instance, speakers, displays, tactile output, holograms and so forth. Examples of input mechanisms 812B might include, for instance, microphones, touchscreens, holograms, cameras, keyboards, mouse or other pointer input, sensors of any type, and so forth.

Embodiments described herein may comprise or utilize a special purpose or general-purpose computing system including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general-purpose or special purpose computing system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: storage media and transmission media.

Computer-readable storage media includes RAM, ROM, EEPROM, CD-ROM, or other optical disk storage, magnetic disk storage, or other magnetic storage devices, or any other physical and tangible storage medium which can be used to store desired program code means in the form of computer-executable instructions or data structures and which can be accessed by a general-purpose or special purpose computing system.

A “network” is defined as one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Transmissions media can include a network and/or data links which can be used to carry desired program code means in the form of computer-executable instructions or data structures and which can be accessed by a general-purpose or special-purpose computing system. Combinations of the above should also be included within the scope of computer-readable media.

Further, upon reaching various computing system components, program code means in the form of computer-executable instructions or data structures can be transferred automatically from transmission media to storage media (or vice versa). For example, computer-executable instructions or data structures received over a network or data link can be buffered in RANI within a network interface module (e.g., a “NIC”), and then eventually transferred to computing system RANI and/or to less volatile storage media at a computing system. Thus, it should be understood that storage media can be included in computing system components that also (or even primarily) utilize transmission media.

Computer-executable instructions comprise, for example, instructions and data which, when executed at a processor, cause a general-purpose computing system, special purpose computing system, or special purpose processing device to perform a certain function or group of functions. Alternatively, or in addition, the computer-executable instructions may configure the computing system to perform a certain function or group of functions. The computer executable instructions may be, for example, binaries or even instructions that undergo some translation (such as compilation) before direct execution by the processors, such as intermediate format instructions such as assembly language, or even source code.

Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computing system configurations, including, personal computers, desktop computers, laptop computers, message processors, handheld devices, multi-processor systems, microprocessor-based or programmable consumer electronics, network PCs, minicomputers, mainframe computers, mobile telephones, PDAs, pagers, routers, switches, data centers, wearables (such as glasses) and the like. The invention may also be practiced in distributed system environments where local and remote computing system, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

The remaining figures may discuss various computing system which may correspond to the computing system 800 previously described. The computing systems of the remaining figures include various components or functional blocks that may implement the various embodiments disclosed herein as will be explained. The various components or functional blocks may be implemented on a local computing system or may be implemented on a distributed computing system that includes elements resident in the cloud or that implement aspect of cloud computing. The various components or functional blocks may be implemented as software, hardware, or a combination of software and hardware. The computing systems of the remaining figures may include more or less than the components illustrated in the figures, and some of the components may be combined as circumstances warrant. Although not necessarily illustrated, the various components of the computing systems may access and/or utilize a processor and memory, such as the at least one processing unit 802 and memory 804, as needed to perform their various functions.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A head-mounted device comprising:
one or more processors;
one or more eye-tracking cameras; and
one or more computer-readable hardware storage devices having stored thereon computer-executable instructions, including a machine-learned artificial intelligence (AI) model, the computer-executable instructions being structured such that, when executed by the one or more processors, the computer-executable instructions configure the head-mounted device to perform at least:
cause the one or more eye-tracking cameras to take a series of images of one or more areas of skin around one or more eyes of a wearer;
use the machine-learned AI model to analyze the series of images to extract a photoplethysmography waveform; and
detect a heart rate based on the photoplethysmography waveform.

2. The head-mounted device of claim 1, wherein at least one of the one or more eye-tracking cameras is an infrared camera.

3. The head-mounted device of claim 1, further comprising one or more infrared light sources configured to emit infrared light at the one or more areas of skin around the one or more eyes of the wearer.

4. The head-mounted device of claim 1, wherein:
the head-mounted device further comprises an inertial measurement unit configured to detect head motion of the wearer; and
the head-mounted device is further configured to remove at least a portion of noise artifacts generated by the head motion from the photoplethysmography waveform.

5. The head-mounted device of claim 4, wherein the inertial measurement unit includes at least one of (1) an accelerometer, (2) a gyroscope, or (3) a magnetometer.

6. The head-mounted device of claim 4, wherein data related to the head motion of the wearer is input into the machine-learned AI model, causing the machine-learned AI model to cancel out at least a portion of noise artifacts generated by the head motion from the photoplethysmography waveform.

7. The head-mounted device of claim 4, the head-mounted device further configured to:
process data generated by the inertial measurement unit to identify one or more frequency bands of the noise artifacts generated by the head motion; and
filter the one or more frequency bands out of the photoplethysmography waveform.

8. The head-mounted device of claim 4, the head-mounted device further configured to:
determine whether a period of time is too noisy based on data generated by the inertial measurement unit during the period; and
in response to determining that the period is too noisy, segment out data generated during the period, including one or more images among the series of images taken during the period.

9. The head-mounted device of claim 8, determining whether the period of time is too noisy, comprising:
determining a standard deviation of values obtained from the inertial measurement unit during the period of time;
when the standard deviation is greater than a predetermined threshold, determining that the period of time is too noisy; and
when the standard deviation is no greater than the predetermined threshold, determining that the period of time is not too noisy.

10. The head-mounted device of claim 1, the head-mounted device further configured to perform a calibration operation to improve the machine-learned AI model based on individual wearers, the calibration operation comprising:
detecting a first set of heart rates of the wearer based on a series of images taken by the one or more eye-tracking cameras and the machine-learned AI model;
detecting a second set of heart rates via a heart rate monitor while the series of images are taken; and
using the second set of heart rates as feedback to calibrate the machine-learned AI model.

11. The head-mounted device of claim 1, wherein:
the head-mounted device further comprises one or more displays configured to display one or more images in front of one or more eyes of the wearer; and
the head-mounted device is further configured to remove at least a portion of noise artifacts generated by the one or more displays from the photoplethysmography waveform.

12. The head-mounted device of claim 11, wherein:
data related to the one or more images displayed on the one or more displays is input into the machine-learned AI model, causing the machine-learned AI model to cancel out at least a portion of noise artifacts generated by the one or more displays.

13. The head-mounted device of claim 11, the head-mounted device further configured to:
determine whether a period of time is too noisy based on data generated by the one or more displays during the period; and
in response to determining that the period is too noisy, segment out data generated during the period, including one or more images among the series of images taken during the period.

14. The head-mounted device of claim 13, determining whether the period of time is too noisy, comprising:
determining a standard deviation of values obtained from the one or more displays during the period of time;
when the standard deviation is greater than a predetermined threshold, determine that a predetermined time window is too noisy; and
when the standard deviation is no greater than the predetermined threshold, determine that the predetermined time window is not too noisy.

15. A method for training an artificial intelligence (AI) model for detecting heart rates based on images taken by one or more eye-tracking cameras of head-mounted devices, the method comprising:
providing a machine learning network configured to train an AI model based on images taken by eye-tracking cameras of head-mounted devices;
taking a plurality of series of images of one or more areas of skin around one or more eyes of a wearer by the one or more eye-tracking cameras of a head-mounted device as training data; and
using the plurality of series of images as training data for the machine learning network to train the AI model in a particular manner, such that the AI model is trained to extract a photoplethysmography waveform and detect a heart rate based on the photoplethysmography waveform.

16. The method of claim 15, wherein:
the machine learning network is an unsupervised network that trains the AI model based on unlabeled image data.

17. The method of claim 15, wherein:
the machine learning network is a supervised network that trains the AI model based on labeled image data, and
the method further comprising:
gathering a plurality of heart rate datasets via a heart rate monitor simultaneously when the plurality of series of images are gathered;
labeling the plurality of series of images with the plurality of heart rate datasets; and
using the plurality of series of images that are labeled with the plurality of heart rate datasets as training data to train the AI model.

18. The method of claim 15, wherein each image in each series of images includes a plurality of pixels;
each pixel corresponds to a color value corresponding; and
the method further comprises:
for each image in each series of images, computing an average value based on color values corresponding to a plurality of pixels in an image; and
the machine learning network to train the AI model configured to extract a photoplethysmography waveform based on average values of images in the plurality of series of images.

19. The method of claim 15, wherein:
the head-mounted device includes an inertial measurement unit configured to detect head motion of the wearer, and
the method further comprises:
gathering a plurality of datasets associated with the head motion of the wearer detected by the inertial measurement unit; and
further using the plurality of datasets associated with the head motion of the wearer as training data in training the AI model, such that the AI model is trained to cancel out at least a portion of noise artifacts generated by head motions.

20. A computer program product comprising one or more hardware storage devices having stored thereon computer-executable instructions including a machine-learned AI model that are structured such that, when the computer-executable instructions are executed by one or more processors of a head-mounted computing system having one or more eye-tracking cameras, the computer-executable instructions configure the head-mounted computing system to perform at least:
cause the one or more eye-tracking cameras to take a series of images of an area of skin around an eye of a wearer;
use the machine-learned AI model to analyze the series of images to extract a photoplethysmography waveform from the series of images; and
detect a heart rate based on the photoplethysmography waveform.
